(19) United States
(12) Patent Application Publication
(10) Pub. No.: US 2005/0129765 A1
(43) Pub. Date: Jun. 16, 2005

(54) Controlled release of topiramate in liquid dosage forms

(76) Inventors: Shaoling Li, Sunnyvale, CA (US); Andrew Lam, South San Francisco, CA (US); Liang Dong, Sunnyvale, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/986,962

(22) Filed: Nov. 12, 2004

Related U.S. Application Data
(60) Provisional application No. 60/519,958, filed on Nov. 14, 2003.

(51) Int. Cl. A61K 9/48; A61K 9/24; A61K 31/7024

(52) U.S. Cl. 424/473; 514/23

(57) ABSTRACT

This invention relates to novel formulations and methods for the controlled release delivery of topiramate; as well as to the use of these formulations and methods for treating disease.

### CROSS-REFERENCE TO RELATED APPLICATIONS

This application claims benefit of U.S. provisional patent application No. 60/519,958, filed Nov. 14, 2003, which is incorporated herein by reference.

### FIELD OF THE INVENTION

This invention relates to novel formulations and methods for the controlled release delivery of topiramate; as well as to the use of these formulations and methods for treating disease.

### BACKGROUND

Topiramate is a well known drug useful in treating a multitude of disorders including epilepsy, tremors, Type II diabetes, mood disorders such as depression, mania, and bipolar disorder, obesity, eating disorders, such as binge eating, post traumatic stress disorder, migraines, cluster headaches, cerebral function disorders, tobacco cessation, and neuropathic pain. In particular, topiramate has been approved as an anti-convulsant drug for the treatment of seizures and seizure disorders such as epilepsy.

Topiramate is a white crystalline powder that is known to be soluble in alkaline solutions containing sodium hydroxide or sodium phosphate, acetone, dimethylsulfoxide and ethanol. In water, topiramate has a solubility of about 9.8 mg/ml. Topiramate is currently sold under the tradename Topamax™ by Ortho-McNeil Pharmacuetical, Inc., Raritan, N.J. Topiramate is available in solid dosage form as a tablet in amounts of 25, 50, 100, 200, 300, and 400 mg.

Conventional oral dosage forms, such as Topamax™, can be described as “immediate-release” dosage forms because, generally, essentially the entire dose of drug is released from the dosage form within a very short period, i.e., minutes, following administration. As this bolus of released drug is absorbed, the plasma drug concentration typically rapidly rises to a maximal or peak so concentration and subsequently declines as the drug is distributed, bound or localized within tissues, biotransformed and/or excreted. The time period for this decline varies for different drugs and depends on many factors but this time period will be characteristic of a particular drug. Generally, during some portion of the time period in which the plasma drug concentration rises, peaks and declines, the drug provides its therapeutic effects, i.e., the plasma drug concentration achieves or exceeds an effective concentration. Moreover, at some point during this time period, the therapeutic effects disappear, i.e., when the plasma drug concentration declines to a level that is below an effective concentration. In addition, often, during a portion of this time surrounding the time the peak concentration is attained, i.e., when the plasma drug concentration is in its highest range, undesired side effects may become apparent.

One focus of efforts to improve drug therapy has been directed to providing non-immediate-release oral drug dosage forms that affect absorption of the drug primarily by altering the release rate of the drug from the dosage form. Osmotic dosage forms, in particular, have been notably successful at providing constant-release of drugs over extended time periods. Osmotic dosage forms, in general, utilize osmotic pressure to generate a driving force for imbibing fluid into a compartment formed, at least in part, by a semipermeable wall that permits free diffusion of fluid but not drug or osmotic agent(s), if present. A substantially constant rate of drug release can be achieved by designing the system to provide a relatively constant osmotic pressure and having suitable exit means for the drug formulation to permit the drug formulation to be released at a rate that corresponds to the rate of fluid imbibed as a result of the relatively constant osmotic pressure. A significant advantage to osmotic systems is that operation is pH-independent and thus continues at the osmotically-determined rate throughout an extended time period even as the dosage form transits the gastrointestinal tract and encounters differing microenvironments having significantly different pH values.

Not every drug, however, can be suitably delivered from these dosage forms because of solubility, metabolic processes, absorption and other physical, chemical and physiological parameters that may be unique to the drug and the mode of delivery. Topiramate, for example, because of its slow hydration rate, especially for the drug-layer formulation with high topiramate content (e.g., drug content >15%), has proven very difficult to be incorporated in an osmotic controlled release solid dosage form. Accordingly, a need exists for improved controlled delivery systems capable of delivering topiramate at a controlled release rate. This invention meets this and other needs.

### SUMMARY

The present invention provides, inter alia, dosage forms for controlled release delivery of topiramate and methods of administering the dosage forms to a subject. The dosage forms can be administered to a subject to treat a disease responsive to topiramate therapy. Diseases responsive to topiramate therapy include, for example, mood disorders such as depression, mania, and bipolar disorder, obesity, eating disorders, such as binge eating, post traumatic stress disorder, migraines, cluster headaches, cerebral function disorders, tobacco cessation, and neuropathic pain.

The dosage forms of the present invention comprise a semipermeable wall, a drug layer, and an expandable layer. The semipermeable wall is permeable to the passage of an exterior biological fluid and substantially impermeable to the passage of drug formulation and surrounds and forms a compartment comprising a plurality of layers. The plurality of layers comprises at least one drug layer comprising topiramate solubilized in a nonaqueous liquid carrier and at least one expandable layer. An orifice in the semipermeable wall connects the exterior of the dosage form and the topiramate formulation for delivering topiramate from the dosage form to the environment. In certain embodiments, the plurality of layers can comprise additional layers such as for example, a barrier layer. In some embodiments, the barrier layer is impermeable to water.

The dosage forms of the present invention, in some embodiments, comprise a plurality of drug layers. For example, in one aspect, the dosage form comprises a second layer comprising topiramate solubilized in a nonaqueous liquid carrier. The second layer can contain the same concentration of topiramate as the first layer or a different concentration of topiramate. In one aspect, the second layer comprises a greater concentration of topiramate than the first layer. The first layer is closer to the core of the dosage form and drug is released successively from the second layer and then from the first layer.

The dosage forms can be arranged in different ways. For example, in certain embodiments, the topiramate layer is encased in a capsule and in outward order from the capsule is a barrier layer, an expandable layer, and the semipermeable wall. In some embodiments, the barrier layer is formed as a coating on the capsule. In some embodiments, the expandable layer is formed as an osmotic layer coated on the barrier layer. The semipermeable wall can be formed as a coating on the osmotic layer. In one aspect, the capsule is a soft capsule. The capsule can comprise a gelatin or non-gelatin hydrophilic polymer.

In other embodiments, the topiramate layer, the barrier layer, and the expandable layer are encased in a capsule, e.g., a hard capsule, the barrier layer separates the topiramate layer from the expandable layer and surrounding the capsule is the semipermeable wall. In one aspect, the expandable layer is formed as an osmotic layer compressed on the barrier layer. In some embodiments, the expandable layer is an osmotic layer. In some embodiments, the expandable layer comprise a fluid-expandable polymer. In certain embodiments, the expandable layer and barrier layer are longitudinally compressed.

The dosage forms of the present invention comprise topiramate solubilized in a nonaqueous liquid carrier. The liquid carrier comprises a lipophilic carrier, a surfactant, or a hydrophilic solvent, or a combination thereof. The hydrophilic solvent can be a liquid polymer such as for example, polyethylene glycol. In some embodiments, the liquid formulation of topiramate is a solution. In other embodiments, the formulation is a suspension. In other embodiments, the liquid formulation is a self-emulsifying formulation. In one aspect, the self-emulsifying formulation is lipid-based.

The dosage forms of the present invention comprise topiramate. In one embodiment, the dosage forms comprise from about 1 mg to about 800 mg of topiramate, preferably from about 1 mg to about 600 mg of topiramate, from about 1 mg to about 300 mg of topiramate, from about 10 mg to about 750 mg of topiramate, from about 10 mg to about 400 mg of topiramate, or from about 25 mg to about 400 mg of topiramate and all combinations as well as specific numerals contained therein. In one aspect of the present invention, the dose of topiramate in the dosage form is, for example, from between about 0.1% to about 60% weight of the dosage form. In one embodiment, the liquid carrier is, for example, between from about 30% to about 50% by weight of the dosage form.

The dosage forms of the present invention comprise topiramate solubilized in a nonaqueous liquid carrier. In certain embodiments, the drug layer comprises from about 10% to about 60% of topiramate and from about 40% to about 90% of the liquid carrier, and all combinations as well as specific percentages contained therein. In one aspect, the drug layer comprises from about 40% to about 60% of topiramate and from about 60% to about 40% of the liquid carrier. In some embodiments, the drug layer comprises about 40% topiramate, about 30% of a surfactant, and about 30% of a hydrophilic solvent. In other embodiments, the drug layer comprises about 60% topiramate, about 20% of a surfactant, and about 20% of a hydrophilic solvent. In one aspect, the surfactant is selected from the group consisting of Cremophor EL and solutol and the hydrophilic solvent is a hydrophilic liquid polymer such as PEG400.

The present also provides methods for controlled release of topiramate comprising orally administering to a subject the dosage forms of the present invention. In one aspect, the release rate of topiramate from the dosage forms is zero order. In another aspect, the release rate of the topiramate from the dosage forms is ascending. In a preferred embodiment, when the release rate is ascending, a hard cap dosage form is used.

### BRIEF DESCRIPTION OF THE DRAWINGS

FIG. 1 illustrates a hard cap dosage form of the present invention.

FIG. 2 illustrates a soft cap dosage form of the present invention.

FIG. 3 illustrates release patterns of topiramate from a dosage form provided by the present invention. A prototype hardcap dosage form (200 mg topiramate) was released in DI water for up to 16 hours. The drug concentration was analyzed by HPLC with RI detector.

FIG. 4 is a prophetic example illustrating the ascending release rate expected for a dosage form comprising multiple layers of drug.

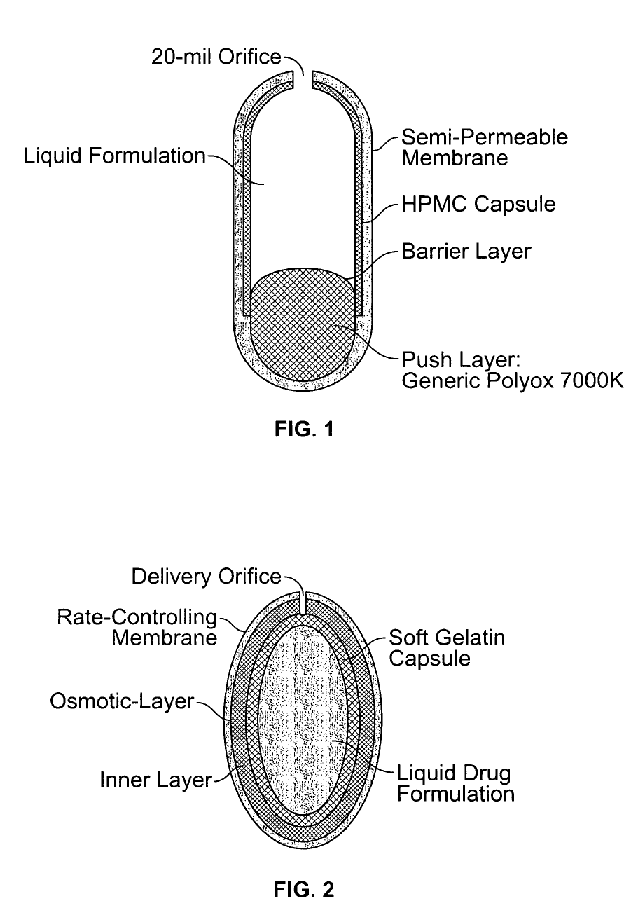

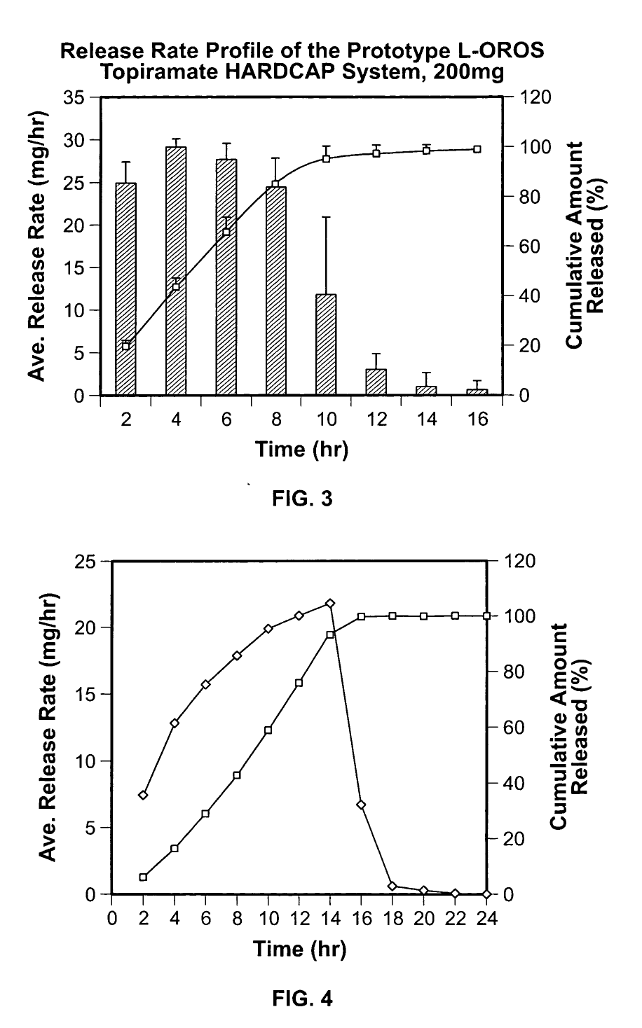

### DETAILED DESCRIPTION

The use of osmotic devices for controlled release delivery of topiramate in solid dosage form suffers from many drawbacks. Osmotic devices for delivering drugs typically comprise at least two component layers within a compartment formed by a semi-permeable wall. One component comprises drug in a mixture with excipients, optionally including osmotically active components, that will form a deliverable drug formulation within the compartment, and the second component layer comprises osmotically active components but does not contain a drug. The osmotically active components in the second component layer typically comprise osmopolymers having relatively large molecular weights and which exhibit “swelling” as fluid is imbibed such that the release of these components through the drug formulation exit means does not occur. As the fluid is imbibed, the osmopolymers swell and push against the deliverable drug formulation of the first component layer to thereby facilitate the release of the drug formulation at a substantially constant rate. These systems require that the drug layer is sufficiently hydrated by the imbibed fluid so that the drug can be effectively extruded from the device and dissolved in the bodily fluids. It has been surprisingly discovered, however, that although topiramate can be effectively administered in solid dosage form through immediate-release orally administered tablets, delivery of solid dosage form of topiramate using osmotic devices is difficult. For example, the delivery of topiramate in amounts of less than 100 mg has proven to be feasible only by incorporating high levels of surfactants in the drug layer to achieve a desirable core hydration rate and a functionally acceptable release rate profile (e.g., approximately 30-50% of surfactant is needed at 1:1.5 and 1:2 of drug to surfactant ratio). Moreover because of topiramate's poor hydration characteristics and the thermal properties of a drug layer comprising high levels of surfactant and topiramate, dosages of greater than 100 mg of topiramate have proven to be difficult to manufacture. A sustained-release oral dosage form of topiramate that can provide substantially constant drug release over an extended period of time would be beneficial for treating many of the diseases and conditions responsive to topiramate therapy. In this regard, it has been surprisingly discovered that the development and use of topiramate in liquid formulation in combination with osmotic devices overcomes the difficulties associated with controlled release delivery of topiramate. In a liquid formulation, controlled release of topiramate at a functionally acceptable release rate profile is possible, i.e., at a zero order or ascending order release rate.

The present invention is, in part, directed to a novel drug layer composition for an osmotic dosage form with therapeutic effects over six, eight, twelve, or twenty-four hours utilizing a single convenient liquid dosage form. The drug layer comprises topiramate in liquid formulation solubilized in a liquid carrier. Preferably, the liquid carrier is nonaqueous. For use in the present invention, a “nonaqueous liquid carrier” can contain a certain amount of aqueous liquid (for example, about 10 or 20% aqueous liquid) as long as the liquid carrier is predominantly nonaqueous. In an exemplary embodiment of the present invention, topiramate can have a solubility of from about 30 mg/ml to about 400 mg/ml, preferably from about 30 mg/ml to about 200 mg/ml in the liquid formulation.

The present invention provides, inter alia, dosage forms capable of delivering high dosages of topiramate to a subject. Topiramate is provided as a liquid formulation in a liquid drug carrier. The liquid formulation can be a solution, suspension, or self-emulsfying formulation. The liquid carrier can be a lipophilic solvent, a surfactant, or a hydrophilic solvent, or a combination thereof. In one embodiment, a lipophilic solvent in combination with one or more surfactants and optionally including one or more hydrophilic solvents is used to formulate topiramate into a liquid formulation. In another embodiment, one or more surfactants are used to formulate topiramate into a liquid formulation. In yet another embodiment, one or more hydrophilic solvents are used to formulate topiramate into a liquid formulation. Accordingly, multi-component or single component liquid carriers can be prepared to formulate the drug, topiramate, into a liquid formulation, including a self-emulsifying formulation.

The present invention also provides, inter alia, a dosage form for releasing topiramate at zero order rate of release or ascending rate of release. An ascending rate of release can be accomplished through the use of a hard cap osmotic device comprising multiple drug layers having various drug concentrations that are released sequentially to provide varying release rates of the active agent.

A drug “release rate” refers to the quantity of drug released from a dosage form per unit time, e.g., milligrams of drug released per hour (mg/hr). Drug release rates are calculated under in vitro dosage form dissolution testing conditions known in the art. As used herein, a drug release rate obtained at a specified time “following administration” refers to the in vitro drug release rate obtained at the specified time following implementation of an appropriate dissolution test. Methods of performing dissolution tests or release rate assays are known in the art. By such tests or assays is meant a standardized assay for the determination of the release rate of a compound from the dosage form tested using a USP Type VII interval release apparatus. It is understood that reagents of equivalent grade can be substituted in the assay in accordance with generally accepted procedures. For example, aliquots of the drug to be tested can be injected into a chromatographic system to quantify the amounts of drug released during the testing intervals, e.g., using a USP Type VII bath indexer immersed in about 50 ml of De-ionized water equilibrated in a constant temperature water bath at 37° C. The time at which a specified percentage of the drug within a dosage form has been released can be referenced as the “Tₓ” value, where “x” is the percent of drug that has been released. A commonly-used reference measurement for evaluating drug release from oral dosage forms is the time at which 70% or 90% of drug within a dosage form has been released. This measurement is referred to as “T₇₀” or “T₉₀” for the dosage form.

An “immediate-release” dose of a drug refers to a dose that is substantially completely released within a time period of about 1 hour or less and, preferably, about 30 minutes or less. Currently, topiramate is marketed in immediate-release form. Osmotic dosage forms such as those of the present invention typically require a short time period following administration in which to become hydrated sufficiently to begin to release drug. In embodiments, wherein the slight delay in initial drug release is not desirable, an immediate-release overcoat can be applied to the surface of the semi-permeable membrane of the dosage form. An immediate-release dose of drug applied as a coating on the surface of a dosage form refers to a dose of drug prepared in a suitable pharmaceutically acceptable carrier to form a coating solution that will dissolve rapidly upon administration thereby providing an immediate-release dose of drug. As is known in the art, such immediate release drug overcoats can contain the same or a different drug or drugs as is contained within the underlying dosage form.

A “periodic release rate” refers to the quantity of drug released from a dosage form during a specified periodic interval as determined at the end of that specified periodic interval, i.e., at each periodic interval when a determination is made, the quantity of drug released represents the periodic release rate during that periodic interval. For example, the quantity of drug released as determined at t=1 h represents the periodic release rate from the dosage form during the first hour following administration and the quantity of drug released as determined at t=2 h represents the periodic release rate during the second hour following administration.

A zero order release rate refers to a constant, linear, continuous, sustained and controlled release rate. An “ascending release rate” refers to a periodic release rate that is increased over the immediately-preceding periodic release rate, where the periodic intervals are the same. For example, when the quantity of drug released from a dosage form is measured at hourly intervals and the quantity of drug released during the fifth hour following administration (determined at t=5 hours) is greater than the quantity of drug released from the dosage form during the fourth hour following administration (determined at t=4 hours), an ascending release rate from the fourth hour to the fifth hour has occurred. It will be appreciated that the first periodic release rate measured, e.g., the periodic release rate at t=1 hour (unless equal to 0), will always be greater than the release rate during the preceding period, e.g., the hour before the dosage form was administered, and, thus, the first periodic release rate always constitutes an occurrence of an ascending release rate. The ascending release rates described herein refer to the release rate from a dosage form adapted to provide sustained release of drug and do not include release of drug from any immediate-release drug coating that can be applied to the dosage form. In dosage form embodiments additionally comprising an immediate-release dose of a drug applied as a coating onto the underlying dosage form, the drug release measured at t=1 hour will generally reflect both the drug released from the immediate-release drug coating and any drug released from the underlying dosage form, however, the quantity of drug released from the drug overcoat is disregarded in determining whether the drug release rate at t=2 hours is greater than the drug release at t=1 hour. In accord with the above-recited definitions, an “ascending release rate over an extended time period” refers to ascending release rates of drug obtained from the time of administration of the dosage form through, and preferably beyond, the mid-point of the relevant T₉₀ for the dosage form. To illustrate, consider a situation where a dosage form has a T₉₀ of about 8 hours. In this situation, an “ascending release rate over an extended time period” is achieved when the release rate at each hour through t=4 hours is greater than the release rate in the immediately-preceding hour. Preferably, the release rate continues to ascend during time periods beyond t=4 hours.

By “sustained release dosage form” is meant a dosage form that releases drug substantially continuously for many hours. Sustained release dosage forms in accord with the present invention exhibit T₉₀ values of at least about 8 to 20 hours and preferably 15 to 18 hours and more preferably about 17 hours or more. The dosage forms continuously release drug for sustained periods of at least about 8 hours, preferably 12 hours or more and, more preferably, 16-20 hours or more. Dosage forms in accord with the present invention exhibit controlled release rates of a therapeutic agent for a prolonged period of time within the sustained release time period.

By “uniform release rate” is meant an average hourly release rate from the core that varies positively or negatively by no more than about 30% and preferably no more than about 25% and most preferably no more than 10% from either the preceding or the subsequent average hourly release rate as determined in a USP Type VII Interval Release Apparatus where the cumulative release is between about 25% to about 75%.

By “prolonged period of time” is meant a continuous period of time of at least about 4 hours, preferably 6-8 hours or more and, more preferably, 10 hours or more. For example, the exemplary osmotic dosage forms described herein generally begin releasing therapeutic agent at a uniform release rate within about 2 to about 6 hours following administration and the uniform rate of release, as defined above, continues for a prolonged period of time from about 25% to until at least about 75% and preferably at least about 85% of the drug is released from the dosage form. Release of therapeutic agent continues thereafter for several more hours although the rate of release is generally slowed somewhat from the uniform release rate.

By “C” is meant the concentration of drug in the blood plasma of a subject, generally expressed as mass per unit volume, typically nanograms per milliliter. For convenience, this concentration can be referred to as “plasma drug concentration” or “plasma concentration” herein which is intended to be inclusive of drug concentration measured in any appropriate body fluid or tissue. The plasma drug concentration at any time following drug administration is referenced as Cₜᵢₘₑ, as in C₉ₕ or C₂₄ₕ.

By “steady state” is meant the condition in which the amount of drug present in the blood plasma of a subject does not vary significantly over a prolonged period of time. A pattern of drug accumulation following continuous administration of a constant dose and dosage form at constant dosing intervals eventually achieves a “steady-state” where the plasma concentration peaks and plasma concentration troughs are essentially identical within each dosing interval. As used herein, the steady-state maximal (peak) plasma drug concentration is referenced as Cₘₐₓ and the minimal (trough) plasma drug concentration is referenced as Cₘᵢₙ. The times following drug administrations at which the steady-state peak plasma and trough drug concentrations occur are referenced as the Tₘₐₓ and the Tₘᵢₙ, respectively.

Persons of skill in the art appreciate that plasma drug concentrations obtained in individual subjects will vary due to interpatient variability in the many parameters affecting drug absorption, distribution, metabolism and excretion. For this reason, unless otherwise indicated, mean values obtained from groups of subjects are used herein for purposes of comparing plasma drug concentration data and for analyzing relationships between in vitro dosage form dissolution rates and in vivo plasma drug concentrations.

By “high dosage” is meant drug loading therapeutic agent topiramate within the dosage form that comprises greater than about 100 mg of topiramate.

The present invention therefore provides, inter alia, both a dosage form and a method for controlled delivery of high doses of topiramate over an extended period of time. In a preferred embodiment, administration of the dosage form will be once a day. This is accomplished through the solubilization of topiramate using liquid formulations. By solubilizing topiramate in a nonaqueous liquid carrier, topiramate can be delivered in a pre-solubilized and more easily absorbed form. Moreover, with topiramate pre-solubilized in a liquid carrier, unlike in its solid dosage form, it can be released even without being hydrated, thereby providing for its controlled release from an osmotic device at an acceptable rate, e.g., a smooth non-erratic release rate.

A liquid formulation of topiramate comprises topiramate and the liquid carrier at varied ratios. Selection of the liquid carrier is based upon drug-excipient compatibility, and physical and chemical stability of the compounds. Specific formulations for use in the present invention will be ascertainable by one skilled in the art using known techniques. Exemplary liquid carriers of the present invention include lipophilic solvents (e.g., oils and lipids), surfactants, and hydrophilic solvents. Exemplary lipophilic solvents, for example, include, but are not limited to, Capmul PG-8, Caprol MPGO, Capryol 90, Plurol Oleique CC 497, Capmul MCM, Labrafac PG, N-Decyl Alcohol, Caprol 10G100, Oleic Acid, Vitamin E, Maisine 35-1, Gelucire 33/01, Gelucire 44/14, Lauryl Alcohol, Captex 355EP, Captex 500, Capylic/Caplic Triglyceride, Peceol, Caprol ET, Labrafil M2125 CS, Labrafac CC, Labrafil M 1944 CS, Captex 8277, Myvacet 9-45, Isopropyl Nyristate, Caprol PGE 860, Olive Oil, Plurol Oleique, Peanut Oil, Captex 300 Low C6, and Capric Acid. Exemplary surfactants for example, include, but are not limited to, Vitamin E TPGS, Cremophor EL-P, Labrasol, Tween 20, Cremophor RH40, Pluronic L-121, Acconon S-35, Pluronic L-31, Pluronic L-35, Pluronic L-44, Tween 80, Pluronic L-64, Solutol HS-15, Span 20, Cremophor EL, Span 80, Pluronic L-43, and Tween 60. Exemplary hydrophilic solvents for example, include, but are not limited to, Isosorbide Dimethyl Ether, Polyethylene Glycol 400 (PEG-3000), Transcutol HP, Polyethylene Glycol 400 (PEG-4000), Polyethylene Glycol 400 (PEG-300), Polyethylene Glycol 400 (PEG-6000), Polyethylene Glycol 400 (PEG-400), Polyethylene Glycol 400 (PEG-8000), Polyethylene Glycol 400 (PEG-600), and Propylene Glycol (PG).

A preferred liquid formulation of topiramate comprises from about 10% to about 60% of topiramate and about 40% to about 90% of one or more liquid carriers. For example, in some embodiments, the liquid formulation will comprise topiramate and a hydrophilic solvent such as PEG400. In such embodiments, the liquid formulation can comprise from about 10% to about 60% of topiramate and about 40% to about 90% of the hydrophilic solvent. In other embodiments, the liquid formulation can comprise about 40% topiramate and about 60% liquid carrier. In one such preferred embodiment, the liquid carrier can comprise about 50% surfactant, such as Cremophor EL, solutol, or Tween 80, and about 50% hydrophilic solvent, such as PEG400. In other exemplary embodiments, the liquid formulation can comprise about 60% topiramate and about 40% liquid carrier. In one such preferred embodiment, the liquid carrier can comprise about 50% surfactant, such as Cremophor EL, or solutol, and about 50% hydrophilic solvent, such as PEG400. The skilled practitioner will understand that any formulation comprising a sufficient dosage of topiramate solubilized in a liquid carrier suitable for administration to a subject and for use in an osmotic device can be used in the present invention. In one exemplary embodiment of the present invention, the liquid carrier is PEG400, Solutol, Cremophor EL, or combination thereof.

The liquid formulation of topiramate can also comprise, for example, additional excipients such as an antioxidant, permeation enhancer and the like. Antioxidants can be provided to slow or effectively stop the rate of any autoxidizable material present in the capsule. Representative antioxidants can comprise a member selected from the group of ascorbic acid; alpha tocopherol; ascorbyl palmitate; ascorbates; isoascorbates; butylated hydroxyanisole; butylated hydroxytoluene; nordihydroguiaretic acid; esters of garlic acid comprising at least 3 carbon atoms comprising a member selected from the group consisting of propyl gallate, octyl gallate, decyl gallate, decyl gallate; 6-ethoxy-2,2,4-trimethyl-1,2-dihydro-guinoline; N-acetyl-2,6-di-t-butyl-p-aminophenol; butyl tyrosine; 3-tertiarybutyl-4-hydroxyanisole; 2-tertiary-butyl-4-hydroxyanisole; 4-chloro-2,6-ditertiary butyl phenol; 2,6-ditertiary butyl p-methoxy phenol; 2,6-ditertiary butyl-p-cresol: polymeric antioxidants; trihydroxybutyro-phenone physiologically acceptable salts of ascorbic acid, erythorbic acid, and ascorbyl acetate; calcium ascorbate; sodium ascorbate; sodium bisulfite; and the like. The amount of antioxidant used for the present purposes, for example, can be about 0.001% to 25% of the total weight of the composition present in the lumen. Antioxidants are known to the prior art in U.S. Pat. Nos. 2,707,154; 3,573,936; 3,637,772; 4,038,434; 4,186,465 and 4,559,237, each of which is hereby incorporated by reference in its entirety for all purposes.

The liquid formulation can comprise permeation enhancers that facilitate absorption of the active agent in the environment of use. Such enhancers can, for example, open the so-called “tight junctions” in the gastrointestinal tract or modify the effect of cellular components, such a p-glycoprotein and the like. Suitable enhancers can include alkali metal salts of salicyclic acid, such as sodium salicylate, caprylic or capric acid, such as sodium caprylate or sodium caprate, and the like. Enhancers can include, for example, the bile salts, such as sodium deoxycholate. Various p-glycoprotein modulators are described in U.S. Pat. Nos. 5,112,817 and 5,643,909, each of which is hereby incorporated by reference in its entirety for all purposes. Various other absorption enhancing compounds and materials are described in U.S. Pat. No. 5,824,638, which also is incorporated herein by reference in its entirety for all purposes. Enhancers can be used either alone or as mixtures in combination with other enhancers.

In certain embodiments, topiramate is administered as a self-emulsifying formulation. Like the other liquid carriers, the surfactant functions to prevent aggregation, reduce interfacial tension between constituents, enhance the free-flow of constituents, and lessen the incidence of constituent retention in the dosage form. The therapeutic emulsion formulation of this invention comprises a surfactant that imparts emulsification. Exemplary surfactants can also include, for example, in addition to the surfactants listed above, a member selected from the group consisting of polyoxyethylenated castor oil comprising 9 moles of ethylene oxide, polyoxyethylenated castor oil comprising 15 moles of ethylene oxide, polyoxyethylene caster oil comprising 20 moles of ethylene oxide, polyoxyethylenated caster oil comprising 25 moles of ethylene oxide, polyoxyethylenated caster oil comprising 40 moles of ethylene oxide, polyoxyethylenated castor oil comprising 52 moles of ethylene oxide, polyoxyethylenated sorbitan monopalmitate comprising 20 moles of ethylene oxide, polyoxyethylenated sorbitan monostearate comprising 20 moles of ethylene oxide, polyoxyethylenated sorbitan monostearate comprising 4 moles of ethylene oxide, polyoxyethylenated sorbitan tristearate comprising 20 moles of ethylene oxide, polyoxyethylenated sorbitan monostearate comprising 20 moles of ethylene oxide, polyoxyethylenated sorbitan trioleate comprising 20 moles of ethylene oxide, polyoxyethylene lauryl ether, polyoxyethylenated stearic acid comprising 40 moles of ethylene oxide, polyoxyethylenated stearic acid comprising 50 moles of ethylene oxide, polyoxyethylenated stearyl alcohol comprising 2 moles of ethylene oxide, and polyoxyethylenated oleyl alcohol comprising 2 moles of ethylene oxide. The surfactants are available from Atlas Chemical Industries.

The drug emulsified formulations of the present invention can initially comprise an oil and a non-ionic surfactant. The oil phase of the emulsion comprises any pharmaceutically acceptable oil which is not immiscible with water. The oil can be an edible liquid such as an non-polar ester of an unsaturated fatty acid, derivatives of such esters, or mixtures of such esters. The oil can be vegetable, mineral, animal or marine in origin. Examples of non-toxic oils can also include, for example, in addition to the surfactants listed above, a member selected from the group consisting of peanut oil, cottonseed oil, sesame oil, corn oil, almond oil, mineral oil, castor oil, coconut oil, palm oil, cocoa butter, safflower, a mixture of mono- and diglycerides of 16 to 18 carbon atoms, unsaturated fatty acids, fractionated triglycerides derived from coconut oil, fractionated liquid triglycerides derived from short chain 10 to 15 carbon atoms fatty acids, acetylated monoglycerides, acetylated diglycerides, acetylated triglycerides, olein known also as glyceral trioleate, palmitin known as glyceryl tripalmitate, stearin known also as glyceryl tristearate, lauric acid hexylester, oleic acid oleylester, glycolyzed ethoxylated glycerides of natural oils, branched fatty acids with 13 molecules of ethyleneoxide, and oleic acid decylester. The concentration of oil, or oil derivative in the emulsion formulation can be from about 1 wt % to about 40 wt %, with the wt % of all constituents in the emulsion preparation equal to 100 wt %. The oils are disclosed in Pharmaceutical Sciences by Remington, 17^{th} Ed., pp. 403-405, (1985) published by Mark Publishing Co., in Encyclopedia of Chemistry, by Van Nostrand Reinhold, 4^{th} Ed., pp. 644-645, (1984) published by Van Nostrand Reinhold Co.; and in U.S. Pat. No. 4,259,323, each of which is incorporated herein be reference in its entirety and for all purposes.

The amount of topiramate incorporated in the dosage forms of the present invention is generally from about 0.1% to about 60% by weight of the composition depending upon the therapeutic indication and the desired administration period, e.g., every 6 hours, every 12 hours, every 24 hours, every 48 hours, and the like. Depending on the dose of drug desired to be administered, one or more of the dosage forms can be administered.

The actual dosage of topiramate will of course vary according to factors such as the type or severity of disease in a subject and particular status of the subject (e.g., the subject's age, size, fitness, extent of symptoms.) as well as other drugs or treatments being administered concurrently. Dosage regimens can be adjusted to provide an optimum therapeutic response. By “therapeutically effective dose” herein is meant a dose that produces effects for which it is administered. More specifically, a therapeutically effective dose of the compound(s) of the invention preferably alleviates symptoms, complications, or biochemical indicia of diseases responsive to topiramate therapy. The exact dose will be ascertainable by one skilled in the art using known techniques (see, e.g., Lieberman, Pharmaceutical Dosage Forms (Vols. 1-3, 1992); Lloyd, 1999, The Art, Science, and Technology of Pharmaceutical Compounding; and Pickar, 1999, Dosage Calculations). A therapeutically effective dose is also one in which any toxic or detrimental side effects of the active agent is outweighed in clinical terms by therapeutically beneficial effects. It is to be further noted that for each particular subject, specific dosage regimens should be evaluated and adjusted over time according to the individual need and professional judgment of the person administering or supervising the administration of the compounds. The dosage forms of the present invention can comprise, for example, from about 1 mg to about 800 mg, 1 mg to about 600 mg, or 1 mg to about 400 mg of topiramate, and all combinations and subcombinations of ranges, as well as specific numerals contained therein. Preferably, a dosage form of the present invention will comprise from about 10 mg to about 300 mg of topiramate, more preferably from about 25 mg to about 200 mg of topiramate.

Diseases or conditions treatable by the methods of the present invention include any disease or condition responsive to topiramate therapy. The list of diseases responsive to topiramate therapy include, but are not limited to, tremor or seizure disorders such as epilepsy, Type II diabetes, mood disorders or affective disorders such as depression, mania, and bipolar disorder, obesity, eating disorders, such as binge eating, post traumatic stress disorder, migraines, cluster headaches, cerebral function disorders, tobacco cessation, and neuropathic pain. In particular, topiramate has been approved as an anti-convulsant drug for the treatment of seizures and seizure disorders such as epilepsy.

As used herein the term “subject” or “patient” refers any mammalian patient or subject to which the compounds of the invention can be administered. In an exemplary embodiment of the present invention, to identify subject patients for treatment according to the methods of the invention, accepted screening methods are employed to determine risk factors associated with a targeted or suspected disease or condition or to determine the status of an existing disease or condition in a subject. These screening methods include, for example, conventional work-ups to determine risk factors that can be associated with the targeted or suspected disease or condition. These and other routine methods allow the clinician to select patients in need of therapy using the methods and formulations of the present invention.

The term “treating” or “treatment” refers to any indicia of success in amelioration of an injury, pathology, or condition, including any objective or subjective parameter such as abatement; remission; diminishing of symptoms or making the injury, pathology, or condition more tolerable to the patient; slowing in the rate of degeneration or decline; making the final point of degeneration less debilitating; or improving a subject's physical or mental well-being. The treatment or amelioration of symptoms can be based on objective or subjective parameters; including the results of a physical examination, neurological examination, and/or psychiatric evaluation. “Treating” or “treatment of a disease or condition responsive to topiramate therapy” includes preventing the onset of symptoms in a subject that may be predisposed to a disease or condition responsive to topiramate therapy but does not yet experience or exhibit symptoms of the disorder (prophylactic treatment), inhibiting the symptoms of the disorder (slowing or arresting its development), providing relief from the symptoms or side-effects of the disorder (including palliative treatment), and/or relieving the symptoms of the disorder (causing regression). Accordingly, the term “treating” includes the administration of the osmotic dosage forms of the present invention to a subject to prevent or delay, to alleviate, or to arrest or inhibit development of the symptoms or conditions associated with diseases or conditions responsive to topiramate therapy, e.g., seizures associated with epilepsy. A skilled medical practitioner will know how to use standard methods to determine whether a patient is suffering from a disease or condition responsive to topiramate therapy.

The term “cerebral function disorder” includes disorders involving intellectual deficits such as senile dementia, Alzheimer's type dementia, memory loss, amnesia/amnesic syndrome, disturbances of consciousness, coma, lowering of attention, speech disorders, Parkinson's disease, autistic disorder, autism, hyperkinetic syndrome, and schizophrenia. Also within the meaning of the term are disorders caused by cerebrovascular diseases (including, but not limited to, cerebral infarction, cerebral bleeding, cerebral arteriosclerosis, cerebral venous thrombosis, head injuries, and the like) where symptoms include disturbance of consciousness, senile dementia, coma, lowering of attention, and speech disorders.

As used herein, the term “seizures” includes but is not limited to, partial seizures, including without limitation: simple partial seizures, complex partial seizures, and secondarily generalized seizures; generalized seizures, including without limitation absence seizures (also called “petit mal”) typical absence seizures, atypical absence seizures, myoclonic seizures, tonic seizures, clonic seizures, generalized tonic-clonic seizures (also called “grand mal”), and atonic seizures; and seizures associated with juvenile myoclonic epilepsy and Lennox-Gastaut syndrome.

The term “neuropathic pain,” as used herein, includes, but is not limited to, neuralgia, trigeminal neurologia, diabetic neuropathy and other forms of nerve damage, allodynia, paraesthesia, hyperaesthesia, phantom pain, phantom limb pain, hyperalgesia, and tinnitus.

As used herein, the term “affective disorder” includes, but is not limited to, manic conditions (e.g., acute mania), manic rapid cycling, bipolar mood disorders or conditions (e.g., manic-depressive bipolar disorder), mood stabilization, post-traumatic stress disorder, depression, anxiety disorders, attention deficit disorder, attention deficit disorder with hyperactivity, compulsive or obsessive-compulsive disorder, narcolepsy, premenstrual syndrome, chronic fatigue syndrome, seasonal affective disorder, substance abuse or addiction, nicotine addiction or craving, and obesity or weight gain.

As used herein, the terms “attention deficit disorder” (ADD), “attention deficit disorder with hyperactivity” (ADDH), and “attention deficit/hyperactivity disorder” (AD/HD), are used in accordance with their accepted meanings in the art. See, e.g., Diagnostic and Statistical Manual of Mental Disorders, Fourth Ed., American Psychiatric Association, 1997 (DSM-IV™); and Diagnostic and Statistical Manual of Mental Disorders, 3d Ed., American Psychiatric Association (1981) (DSM-III™).

As used herein and unless otherwise indicated, the term “depression” includes a disease or condition characterized by changes in mood, feelings of intense sadness, despair, mental slowing, loss of concentration, pessimistic worry, agitation, and self-deprecation. Physical symptoms of depression that can be reduced or alleviated by the methods of the invention include, but are not limited to, insomnia, anorexia, weight loss, decreased energy and libido, and abnormal hormonal circadian rhythms.

As used herein and unless otherwise indicated, the term “cluster headache” includes, but is not limited to, migrainous neuralgia, chronic migrainous neuralgia, erythroprosopalgia, Raeder's syndrome, spenopalatine neuralgia, ciliary neuralgia, vidian neuralgia, histamine cephalalgia, episodic cluster headache, and chronic cluster headache.

In some embodiments, the oral dosage forms of the present invention will be administered either singly or concomitantly with at least one other therapy or therapeutic agent, e.g., with other anticonvulsant drugs, neuroprotective drugs, antipsychotics, antidepressants, and the like. “Concomitant administration” of a known drug with a dosage form of the present invention means administration of the drug and the dosage form at such time that both the known drug and the dosage form will have a therapeutic effect.

Topiramate is widely available and can be prepared using the processes described in U.S. Pat. Nos. 4,513,600, 4,513,006, and 5,387,700, each of which is hereby incorporated by reference in its entirety for all purposes. Topiramate is a sulfamate-substituted monosaccharide having the chemical name 2,3:4,5-di-O-isopropylidene-β-D-fructopyranose sulfamate. The molecular formula is C₁₂H₂₁NO₈S. The term topiramate as used herein, refers to 3:4,5-di-O-isopropylidene-β-D-fructopyranose sulfamate and isomers and mixtures of isomers thereof. As used herein, the term topiramate refers to topiramate weak acid.

The present invention provides a sustained release liquid formulation of topiramate for use with oral osmotic devices. Oral osmotic devices for delivering liquid formulations and methods of using them are known in the art, for example, as described and claimed in the following U.S. patents owned by ALZA corporation: U.S. Pat. Nos. 6,419,952; 6,174,547; 6,551,613; 5,324,280; 4,111,201; and 6,174,547; each of which is hereby incorporated by reference in its entirety for all purposes. Methods of using oral osmotic devices for delivering therapeutic agents at an ascending rate of release can be found in International Application Numbers WO 98/06380, WO 98/23263, and WO 99/62496, each of which is hereby incorporated by reference in its entirety for all purposes.

The osmotic dosage forms of the present invention can possess two distinct forms, a soft capsule form and a hard capsule form. The soft capsule, as used by the present invention, preferably in its final form comprises one piece. The one-piece capsule is of a sealed construction encapsulating the drug formulation therein. The capsule can be made by various processes including the plate process, the rotary die process, the reciprocating die process, and the continuous process. An example of the plate process is as follows. The plate process uses a set of molds. A warm sheet of a prepared capsule lamina-forming material is laid over the lower mold and the formulation poured on it. A second sheet of the lamina-forming material is placed over the formulation followed by the top mold. The mold set is placed under a press and a pressure applied, with or without heat to form a unit, capsule. The capsules are washed with a solvent for removing excess agent formulation from the exterior of the capsule, and the air-dried capsule is capsuled with a semipermeable wall. The rotary die process uses two continuous films of capsule lamina-forming material that are brought into convergence between a pair of revolving dies and an injector wedge. The process fills and seals the capsule in dual and coincident operations. In this process, the sheets of capsule lamina-forming material are fed over guide rolls, and then down between the wedge injector and the die rolls. The agent formulation to be capsuled flows by gravity into a positive displacement pump. The pump meters the agent formulation through the wedge injector and into the sheets between the die rolls. The bottom of the wedge contains small orifices lined up with the die pockets of the die rolls. The capsule is about half-sealed when the pressure of pumped agent formulation forces the sheets into the die pockets, wherein the capsules are simultaneously filled, shaped, hermetically sealed and cut from the sheets of lamina-forming materials. The sealing of the capsule is achieved by mechanical pressure on the die rolls and by heating of the sheets of lamina-forming materials by the wedge. After manufacture, the agent formulation-filled capsules are dried in the presence of forced air, and a semipermeable lamina capsuled thereto.

The reciprocating die process produces capsules by leading two films of capsule lamina-forming material between a set of vertical dies. The dies as they close, open, and close perform as a continuous vertical plate forming row after row of pockets across the film. The pockets are filled with agent formulation, and as the pockets move through the dies, they are sealed, shaped, and cut from the moving film as capsules filled with agent formulation. A semipermeable capsulating lamina is coated thereon to yield the capsule. The continuous process is a manufacturing system that also uses rotary dies, with the added feature that the process can successfully fill active agent in dry powder form into a soft capsule, in addition to encapsulating liquids. The filled capsule of the continuous process is encapsulated with a semipermeable polymeric material to yield the capsule. Procedures for manufacturing soft capsules are disclosed in U.S. Pat. No. 4,627,850 and U.S. Pat. No. 6,419,952, each of which is hereby incorporated by reference in its entirety for all purposes.

The dosage forms of the present invention can also be made from an injection-moldable composition by an injection-molding technique. Injection-moldable compositions provided for injection-molding into the semipermeable wall comprise a thermoplastic polymer, or the compositions comprise a mixture of thermoplastic polymers and optional injection-molding ingredients. The thermoplastic polymer that can be used for the present purpose comprise polymers that have a low softening point, for example, below 200° C., preferably within the range of 40° C. to 180° C. The polymers, are preferably synthetic resins, addition polymerized resins, such as polyamides, resins obtained from diepoxides and primary alkanolamines, resins of glycerine and phthalic anhydrides, polymethane, polyvinyl resins, polymer resins with end-positions free or esterified carboxyl or caboxamide groups, for example with acrylic acid, acrylic amide, or acrylic acid esters, polycaprolactone, and its copolymers with dilactide, diglycolide, valerolactone and decalactone, a resin composition comprising polycaprolactone and polyalkylene oxide, and a resin composition comprising polycaprolactone, a polyalkylene oxide such as polyethylene oxide, poly(cellulose) such as poly(hydroxypropylmethylcellulose), poly(hydroxyethylmethylcellulose), and poly(hydroxypropylcellulose). The membrane forming composition can comprise optional membrane-forming ingredients such as polyethylene glycol, talcum, polyvinylalcohol, lactose, or polyvinyl pyrrolidone. The compositions for forming an injection-molding polymer composition can comprise 100% thermoplastic polymer. The composition in another embodiment comprises 10% to 99% of a thermoplastic polymer and 1% to 90% of a different polymer with the total equal to 100%. The invention provides also a thermoplastic polymer composition comprising 1% to 98% of a first thermoplastic polymer, 1% to 90% of a different, second polymer and 1% to 90% of a different, third polymer with all polymers equal to 100%. Representation composition comprises 20% to 90% of thermoplastic polycaprolactone and 10% to 80% of poly(alkylene oxide); a composition comprising 20% to 90% polycaprolactone and 10% to 60% of poly(ethylene oxide) with the ingredients equal to 100%; a composition comprising 10% to 97% of polycaprolactone, 10% to 97% poly(alkylene oxide), and 1% to 97% of poly(ethylene glycol) with all ingredients equal to 100%; a composition comprising 20% to 90% polycaprolactone and 10% to 80% of poly(hydroxypropylcellulose) with all ingredients equal to 100%; and a composition comprising 1% to 90% polycaprolactone, 1% to 90% poly(ethylene oxide), 1% to 90% poly(hydroxypropylcellulose) and 1% to 90% poly(ethylene glycol) with all ingredients equal to 100%. The percent, expressed is weight percent wt %.

In another embodiment of the invention, a composition for injection-molding to provide a membrane can be prepared by blending a composition comprising a polycaprolactone 63 wt %, polyethylene oxide 27 wt %, and polyethylene glycol 10 wt % in a conventional mixing machine, such as a Moriyama™ Mixer at 65° C. to 95° C., with the ingredients added to the mixer in the following addition sequence, polycaprolactone, polyethylene oxide and polyethylene glycol. In one example, all the ingredients are mixed for 135 minutes at a rotor speed of 10 to 20 rpm. Next, the blend is fed to a Baker Perkins Kneader™ extruder at 80° C. to 90° C., at a pump speed of 10 rpm and a screw speed of 22 rpm, and then cooled to 10° C. to 12° C., to reach a uniform temperature. Then, the cooled extruded composition is fed to an Albe Pelletizer, converted into pellets at 250° C., and a length of 5 mm. The pellets next are fed into an injection-molding machine, an Arburg Allrounder™ at 200° F. to 350° C. (93° C. to 177° C.), heated to a molten polymeric composition, and the liquid polymer composition forced into a mold cavity at high pressure and speed until the mold is filled and the composition comprising the polymers are solidified into a preselected shape. The parameters for the injection-molding consists of a band temperature through zone 1 to zone 5 of the barrel of 195° F. (91° C.) to 375° F., (191° C.), an injection-molding pressure of 1818 bar, a speed of 55 cm³/s, and a mold temperature of 75° C. The injection-molding compositions and injection-molding procedures are disclosed in U.S. Pat. No. 5,614,578, herein incorporated by reference in its entirety and for all purposes.

Alternatively, the capsule can be made conveniently in two parts, with one part (the “cap”) slipping over and capping the other part (the “body”) as long as the capsule is deformable under the forces exerted by the expandable layer and seals to prevent leakage of the liquid, active agent formulation from between the telescoping portions of the body and cap. The two parts completely surround and capsulate the internal lumen that contains the liquid, active agent formulation, which can contain useful additives. The two parts can be fitted together after the body is filled with a preselected formulation. The assembly can be done by slipping or telescoping the cap section over the body section, and sealing the cap and body, thereby completely surrounding and encapsulating the formulation of active agent.

Soft capsules typically have a wall thickness that is greater than the wall thickness of hard capsules. For example, soft capsules can, for example, have a wall thickness on the order of 10-40 mils, about 20 mils being typical, whereas hard capsules can, for example, have a wall thickness on the order of 2-6 mils, about 4 mils being typical.

In one embodiment of the dosage system, a soft capsule can be of single unit construction and can be surrounded by an unsymmetrical hydro-activated layer as the expandable layer. The expandable layer will generally be unsymmetrical and have a thicker portion remote from the exit orifice. As the hydro-activated layer imbibes and/or absorbs external fluid, it expands and applies a push pressure against the wall of capsule and optional barrier layer and forces active agent formulation through the exit orifice. The presence of an unsymmetrical layer functions to assure that the maximum dose of agent is delivered from the dosage form, as the thicker section of layer distant from passageway swells and moves towards the orifice.

In yet another configuration, the expandable layer can be formed in discrete sections that do not entirely encompass an optionally barrier layer-coated capsule. The expandable layer can be a single element that is formed to fit the shape of the capsule at the area of contact. The expandable layer can be fabricated conveniently by tableting to form the concave surface that is complementary to the external surface of the barrier-coated capsule. Appropriate tooling such as a convex punch in a conventional tableting press can provide the necessary complementary shape for the expandable layer. In this case, the expandable layer is granulated and compressed, rather than formed as a coating. The methods of formation of an expandable layer by tableting are well known, having been described, for example in U.S. Pat. Nos. 4,915,949; 5,126,142; 5,660,861; 5,633,011; 5,190,765; 5,252,338; 5,620,705; 4,931,285; 5,006,346; 5,024,842; and 5,160,743, each of which is hereby incorporated by reference in its entirety for all purposes.

In some embodiments, a barrier layer can be first coated onto the capsule and then the tableted, expandable layer is attached to the barrier-coated capsule with a biologically compatible adhesive. Suitable adhesives include, for example, starch paste, aqueous gelatin solution, aqueous gelatin/glycerin solution, acrylate-vinylacetate based adhesives such as Duro-Tak adhesives (National Starch and Chemical Company), aqueous solutions of water soluble hydrophilic polymers such as hydroxypropyl methyl cellulose, hydroxymethyl cellulose, hydroxyethyl cellulose, and the like. That intermediate dosage form can be then coated with a semipermeable layer. The exit orifice is formed in the side or end of the capsule opposite the expandable layer section. As the expandable layer imbibes fluid, it will swell. Since it is constrained by the semipermeable layer, as it expands it will compress the barrier-coated capsule and express the liquid, active agent formulation from the interior of the capsule into the environment of use.

The hard capsules are typically composed of two parts, a cap and a body, which are fitted together after the larger body is filled with a preselected appropriate formulation. This can be done by slipping or telescoping the cap section over the body section, thus completely surrounding and encapsulating the useful agent formulation. Hard capsules can be made, for example, by dipping stainless steel molds into a bath containing a solution of a capsule lamina-forming material to coat the mold with the material. Then, the molds are withdrawn, cooled, and dried in a current of air. The capsule is stripped from the mold and trimmed to yield a lamina member with an internal lumen. The engaging cap that telescopically caps the formulation receiving body is made in a similar manner. Then, the closed and filled capsule can be capsuled with a semipermeable lamina. The semipermeable lamina can be applied to capsule parts before or after parts and are joined into the final capsule. In another embodiment, the hard capsules can be made with each part having matched locking rings near their opened end that permit joining and locking together the overlapping cap and body after filling with formulation. In this embodiment, a pair of matched locking rings are formed into the cap portion and the body portion, and these rings provide the locking means for securely holding together the capsule. The capsule can be manually filled with the formulation, or they can be machine filled with the formulation. In the final manufacture, the hard capsule is capsuled with a semipermeable lamina permeable to the passage of fluid and substantially impermeable to the passage of useful agent. Methods of forming hard cap dosage forms are described in U.S. Pat. No. 6,174,547, U.S. Pat. Nos. 6,596,314, 6,419,952, and 6,174,547, each of which is incorporated herein by reference in its entirety and for all purposes.

The hard and soft capsules can comprise, for example, gelatin; gelatin having a viscosity of 15 to 30 millipoises and a bloom strength up to 150 grams; gelatin having a bloom value of 160 to 250; a composition comprising gelatin, glycerine, water and titanium dioxide; a composition comprising gelatin, erythrosin, iron oxide and titanium dioxide; a composition comprising gelatin, glycerine, sorbitol, potassium sorbate and titanium dioxide; a composition comprising gelatin, acacia glycerine, and water; and the like. Materials useful for forming capsule wall are known in U.S. Pat. No. 4,627,850; and in 4,663,148, each of which is hereby incorporated by reference in its entirety for all purposes. Alternatively, the capsules can be made out of materials other than gelatin (see for example, products made by BioProgres plc).

The capsules typically can be provided, for example, in sizes from about 3 to about 22 minims (1 minimim being equal to 0.0616 ml) and in shapes of oval, oblong or others. They can be provided in standard shape and various standard sizes, conventionally designated as (000), (00), (0), (1), (2), (3), (4), and (5). The largest number corresponds to the smallest size. Non-standard shapes can be used as well. In either case of soft capsule or hard capsule, non-conventional shapes and sizes can be provided if required for a particular application.

The osmotic devices of the present invention comprise a semipermeable wall permeable to the passage of exterior biological fluid and substantially impermeable to the passage of drug formulation. The selectively permeable composition used for forming the wall are essentially non-erodible and they are insoluble in biological fluids during the life of the osmotic system. The semipermeable wall comprises a composition that does not adversely affect the host, the drug formulation, an osmopolymer, osmogent and the like. Representative polymers for forming semipermeable wall comprise semipermeable homopolymers, semipermeable copolymers, and the like. In one presently preferred embodiment, the compositions can comprise cellulose esters, cellulose ethers, and cellulose ester-ethers. The cellulosic polymers typically have a degree of substitution, “D.S.”, on their anhydroglucose unit from greater than 0 up to 3 inclusive. By degree of substitution is meant the average number of hydroxyl groups originally present on the anhydroglucose unit that are replaced by a substituting group, or converted into another group. The anhydroglucose unit can be partially or completely substituted with groups such as acyl, alkanoyl, alkenoyl, aroyl, alkyl, alkoxy, halogen, carboalkyl, alkylcarbamate, alkylcarbonate, alkylsulfonate, alkylsulfamate, semipermeable polymer forming groups, and the like. The semipermeable compositions typically include a member selected from the group consisting of cellulose acylate, cellulose diacylate, cellulose triacylate, cellulose triacetate, cellulose acetate, cellulose diacetate, cellulose triacetate, mono-, di- and tri-cellulose alkanylates, mono-, di-, and tri-alkenylates, mono-, di-, and tri-aroylates, and the like. Exemplary polymers can include, for example, cellulose acetate have a D.S. of 1.8 to 2.3 and an acetyl content of 32 to 39.9%; cellulose diacetate having a D.S. of 1 to 2 and an acetyl content of 21 to 35%, cellulose triacetate having a D.S. of 2 to 3 and an acetyl content of 34 to 44.8%, and the like. More specific cellulosic polymers include cellulose propionate having a D.S. of 1.8 and a propionyl content of 38.5%; cellulose acetate propionate having an acetyl content of 1.5 to 7% and an acetyl content of 39 to 42%; cellulose acetate propionate having an acetyl content of 2.5 to 3%, an average propionyl content of 39.2 to 45%, and a hydroxyl content of 2.8 to 5.4%; cellulose acetate butyrate having a D.S. of 1.8, an acetyl content of 13 to 15%, and a butyryl content of 34 to 39%; cellulose acetate butyrate having an acetyl content of 2 to 29%, a butyryl content of 17 to 53%, and a hydroxyl content of 0.5 to 4.7%; cellulose triacylates having a D.S. of 2.6 to 3 such as cellulose trivalerate, cellulose trilamate, cellulose tripalmitate, cellulose trioctanoate, and cellulose tripropionate; cellulose diesters having a D.S. of 2.2 to 2.6 such as cellulose disuccinate, cellulose dipalmitate, cellulose dioctanoate, cellulose dicarpylate, and the like; mixed cellulose esters such as cellulose acetate valerate, cellulose acetate succinate, cellulose propionate succinate, cellulose acetate octanoate, cellulose valerate palmitate, cellulose acetate heptonate, and the like. Semipermeable polymers are known in U.S. Pat. No. 4,077,407 and they can be synthesized by procedures described in Encyclopedia of Polymer Science and Technology, Vol. 3, pages 325 to 354, 1964, published by Interscience Publishers, Inc., New York; each of which is hereby incorporated by reference in its entirety for all purposes. Additional semipermeable polymers for forming the semipermeable wall can comprise, for example, cellulose acetaldehyde dimethyl acetate; cellulose acetate ethylcarbamate; cellulose acetate methylcarbamate; cellulose dimethylaminoacetate; semipermeable polyamide; semipermeable polyurethanes; semipermeable sulfonated polystyrenes; cross-linked selectively semipermeable polymers formed by the coprecipitation of a polyanion and a polycation as disclosed in U.S. Pat. Nos. 3,173,876; 3,276,586; 3,541,005; 3,541,006; and 3,546,142, each of which is hereby incorporated by reference in its entirety for all purposes; semipermeable polymers as disclosed in U.S. Pat. No. 3,133,132, hereby incorporated by reference in its entirety for all purposes; semipermeable polystyrene derivatives; semipermeable poly (sodium styrenesulfonate); semipermeable poly (vinylbenzyltremethylammonium chloride); semipermeable polymers, exhibiting a fluid permeability of 10⁻⁵ to 10⁻² (cc. mil/cm hr.atm) expressed as per atmosphere of hydrostatic or osmotic pressure differences across a semipermeable wall. The polymers are known to the art in U.S. Pat. Nos. 3,845,770; 3,916,899; and 4,160,020; and in Handbook of Common Polymers, by Scott, J. R., and Roff, W. J., 1971, published by CRC Press, Cleveland. Ohio, each of which is hereby incorporated by reference in its entirety for all purposes.

The semipermeable wall can also comprise a flux regulating agent. The flux regulating agent is a compound added to assist in regulating the fluid permeability or flux through the wall. The flux regulating agent can be a flux enhancing agent or a decreasing agent. The agent can be preselected to increase or decrease the liquid flux. Agents that produce a marked increase in permeability to fluids such as water are often essentially hydrophilic, while those that produce a marked decrease to fluids such as water are essentially hydrophobic. The amount of regulator in the wall when incorporated therein generally is from about 0.01% to 20% by weight or more. The flux regulator agents in one embodiment that increase flux include, for example, polyhydric alcohols, polyalkylene glycols, polyalkylenediols, polyesters of alkylene glycols, and the like. Typical flux enhancers include polyethylene glycol 300, 400, 600, 1500, 4000, 6000, poly(ethylene glycol-co-propylene glycol), and the like; low molecular weight gylcols such as polypropylene glycol, polybutylene glycol and polyamylene glycol: the polyalkylenediols such as poly(1,3-propanediol), poly(1,4-butanediol), poly(1,6-hexanediol), and the like; aliphatic diols such as 1,3-butylene glycol, 1,4-pentamethylene glycol, 1,4-hexamethylene glycol, and the like; alkylene triols such as glycerine, 1,2,3-butanetriol, 1,2,4-hexanetriol, 1,3,6-hexanetriol and the like; esters such as ethylene glycol dipropionate, ethylene glycol butyrate, butylene glucol dipropionate, glycerol acetate esters, and the like. Representative flux decreasing agents include, for example, phthalates substituted with an alkyl or alkoxy or with both an alkyl and alkoxy group such as diethyl phthalate, dimethoxyethyl phthalate, dimethyl phthalate, and [di(2-ethylhexyl)phthalate], aryl phthalates such as triphenyl phthalate, and butyl benzyl phthalate; insoluble salts such as calcium sulphate, barium sulphate, calcium phosphate, and the like; insoluble oxides such as titanium oxide; polymers in powder, granule and like form such as polystyrene, polymethylmethacrylate, polycarbonate, and polysulfone; esters such as citric acid esters esterfied with long chain alkyl groups; inert and substantially water impermeable fillers; resins compatible with cellulose based wall forming materials, and the like. Other materials that can be used to form the semipermeable wall for imparting flexibility and elongation properties to the wall, for making the wall less-to-nonbrittle and to render tear strength, include, for example, phthalate plasticizers such as dibenzyl phthalate, dihexyl phthalate, butyl octyl phthalate, straight chain phthalates of six to eleven carbons, di-isononyl phthalte, di-isodecyl phthalate, and the like. The plasticizers include nonphthalates such as triacetin, dioctyl azelate, epoxidized tallate, tri-isoctyl trimellitate, tri-isononyl trimellitate, sucrose acetate isobutyrate, epoxidized soybean oil, and the like. The amount of plasticizer in a wall when incorporated therein is about 0.01% to 20% weight, or higher.

The semipermeable wall surrounds and forms a compartment containing a plurality of layers, one of which is an expandable layer which in some embodiments, can contain osmotic agents. The expandable layer comprises in one embodiment a hydroactivated composition that swells in the presence of water, such as that present in gastric fluids. Conveniently, it can comprise an osmotic composition comprising an osmotic solute that exhibits an osmotic pressure gradient across the semipermeable layer against an external fluid present in the environment of use. In another embodiment, the hydro-activated layer comprises a hydrogel that imbibes and/or absorbs fluid into the layer through the outer semipermeable wall. The semipermeable wall is non-toxic. It maintains its physical and chemical integrity during operation and it is essentially free of interaction with the expandable layer.

The expandable layer in one preferred embodiment comprises a hydroactive layer comprising a hydrophilic polymer, also known as osmopolymers. The osmopolymers exhibit fluid imbibition properties. The osmopolymers are swellable, hydrophilic polymers, which osmopolymers interact with water and biological aqueous fluids and swell or expand to an equilibrium state. The osmopolymers exhibit the ability to swell in water and biological fluids and retain a significant portion of the imbibed fluid within the polymer structure. The osmopolymers swell or expand to a very high degree, usually exhibiting a 2 to 50 fold volume increase. The osmopolymers can be noncross-linked or cross-linked. The swellable, hydrophilic polymers are in one embodiment lightly cross-linked, such cross-links being formed by covalent or ionic bonds or residue crystalline regions after swelling. The osmopolymers can be of plant, animal or synthetic origin.

The osmopolymers are hydrophilic polymers. Hydrophilic polymers suitable for the present purpose include poly (hydroxy-alkyl methacrylate) having a molecular weight of from 30,000 to 5,000,000; poly (vinylpyrrolidone) having a molecular weight of from 10,000 to 360,000; anionic and cationic hydrogels; polyelectrolytes complexes; poly (vinyl alcohol) having a low acetate residual, cross-linked with glyoxal, formaldehyde, or glutaraldehyde and having a degree of polymerization of from 200 to 30,000; a mixture of methyl cellulose, cross-linked agar and carboxymethyl cellulose; a mixture of hydroxypropyl methylcellulose and sodium carboxymethylcellulose; a mixture of hydroxypropyl ethylcellulose and sodium carboxymethyl cellulose, a mixture of sodium carboxymethylcellulose and methylcellulose, sodium carboxymethylcellulose; potassium carboxymethylcellulose; a water insoluble, water swellable copolymer formed from a dispersion of finely divided copolymer of maleic anhydride with styrene, ethylene, propylene, butylene or isobutylene crosslinked with from 0.001 to about 0.5 moles of saturated cross-linking agent per mole of maleic anhydride per copolymer; water swellable polymers of N-vinyl lactams; polyoxyethylene-polyoxypropylene gel; carob gum; polyacrylic gel; polyester gel; polyuria gel; polyether gel, polyamide gel; polycellulosic gel; polygum gel; initially dry hydrogels that imbibe and absorb water which penetrates the glassy hydrogel and lowers its glass temperature; and the like.

Representative of other osmopolymers can comprise polymers that form hydrogels such as Carbopol™. acidic carboxypolymer, a polymer of acrylic acid cross-linked with a polyallyl sucrose, also known as carboxypolymethylene, and carboxyvinyl polymer having a molecular weight of 250,000 to 4,000,000; Cyanamer™ polyacrylamides; cross-linked water swellable indenemaleic anhydride polymers; Good-rite™ polyacrylic acid having a molecular weight of 80,000 to 200,000; Polyox™ polyethylene oxide polymer having a molecular weight of 100,000 to 5,000,000 and higher; starch graft copolymers; Aqua-Keeps™ acrylate polymer polysaccharides composed of condensed glucose units such as diester cross-linked polygluran; and the like. Representative polymers that form hydrogels are known to the prior art in U.S. Pat. No. 3,865,108; U.S. Pat. No. 4,002,173; U.S. Pat. No. 4,207,893; and in Handbook of Common Polymers, by Scott and Roff, published by the Chemical Rubber Co., Cleveland, Ohio, each of which is hereby incorporated by reference in its entirety for all purposes. The amount of osmopolymer comprising a hydro-activated layer can be from about 5% to 100%.

The expandable layer in another manufacture can comprise an osmotically effective compound that comprises inorganic and organic compounds that exhibit an osmotic pressure gradient across a semipermeable wall against an external fluid. The osmotically effective compounds, as with the osmopolymers, imbibe fluid into the osmotic system, thereby making available fluid to push against the inner wall, i.e., in some embodiments, the barrier layer and/or the wall of the soft or hard capsule for pushing active agent from the dosage form. The osmotically effective compounds are known also as osmotically effective solutes, and also as osmagents. Osmotically effective solutes that can be used comprise magnesium sulfate, magnesium chloride, potassium sulfate, sodium sulfate, lithium sulfate, potassium acid phosphate, mannitol, urea, inositol, magnesium succinate, tartaric acid, carbohydrates such as raffinose, sucrose, glucose, lactose, sorbitol, and mixtures therefor. The amount of osmagent in can be from about 5% to 100% of the weight of the layer. The expandable layer optionally comprises an osmopolymer and an osmagent with the total amount of osmopolymer and osmagent equal to 100%. Osmotically effective solutes are known to the prior art as described in U.S. Pat. No. 4,783,337, incorporated herein by reference in its entirety for all purposes.

In certain embodiments, the dosage forms further can comprise a barrier layer. The barrier layer in certain embodiments is deformable under the pressure exerted by the expandable layer and will be impermeable (or less permeable) to fluids and materials that can be present in the expandable layer, the liquid active agent formulation and in the environment of use, during delivery of the active agent formulation. A certain degree of permeability of the barrier layer can be permitted if the delivery rate of the active agent formulation is not detrimentally effected. However, it is preferred that barrier layer not completely transport through it fluids and materials in the dosage form and the environment of use during the period of delivery of the active agent. The barrier layer can be deformable under forces applied by expandable layer so as to permit compression of capsule to force the liquid, active agent formulation from the exit orifice. In some embodiments, the barrier layer will be deformable to such an extent that it create a seal between the expandable layer and the semipermeable layer in the area where the exit orifice is formed. In that manner, the barrier layer will deform or flow to a limited extent to seal the initially, exposed areas of the expandable layer and the semipermeable layer when the exit orifice is being formed, such as by drilling or the like, or during the initial stages of operation. When sealed, the only avenue for liquid permeation into the expandable layer is through the semipermeable layer, and there is no back-flow of fluid into the expandable layer through the exit orifice.

Suitable materials for forming the barrier layer can include, for example, polyethylene, polystyrene, ethylene-vinyl acetate copolymers, polycaprolactone and Hytrel™ polyester elastomers (Du Pont), cellulose acetate, cellulose acetate pseudolatex (such as described in U.S. Pat. No. 5,024,842), cellulose acetate propionate, cellulose acetate butyrate, ethyl cellulose, ethyl cellulose pseudolatex (such as Surelease™ as supplied by 10 Colorcon, West Point, Pa. or Aquacoat™ as supplied by FMC Corporation, Philadelphia, Pa.), nitrocellulose, polylactic acid, poly-glycolic acid, polylactide glycolide copolymers, collagen, polyvinyl alcohol, polyvinyl acetate, polyethylene vinylacetate, polyethylene teraphthalate, polybutadiene styrene, polyisobutylene, polyisobutylene isoprene copolymer, polyvinyl chloride, polyvinylidene chloride-vinyl chloride copolymer, copolymers of acrylic acid and methacrylic acid esters, copolymers of methylmethacrylate and ethylacrylate, latex of acrylate esters (such as Eudragit™ supplied by RohmPharma, Darmstaat, Germany), polypropylene, copolymers of propylene oxide and ethylene oxide, propylene oxide ethylene oxide block copolymers, ethylenevinyl alcohol copolymer, polysulfone, ethylene vinylalcohol copolymer, polyxylylenes, polyalkoxysilanes, polydimethyl siloxane, polyethylene glycol-silicone elastomers, electromagnetic irradiation crosslinked acrylics, silicones, or polyesters, thermally crosslinked acrylics, silicones, or polyesters, butadiene-styrene rubber, and blends of the above.

Preferred materials can include cellulose acetate, copolymers of acrylic acid and methacrylic acid esters, copolymers of methylmethacrylate and ethylacrylate, and latex of acrylate esters. Preferred copolymers can include poly (butyl methacrylate), (2-dimethylaminoethyl)methacrylate, methyl methacrylate) 1:2:1, 150,000, sold under the trademark EUDRAGIT E; poly (ethyl acrylate, methyl methacrylate) 2:1, 800,000, sold under the trademark EUDRAGIT NE 30 D; poly (methacrylic acid, methyl methacrylate) 1:1, 135,000, sold under the trademark EUDRAGIT L; poly (methacrylic acid, ethyl acrylate) 1:1, 250,000, sold under the trademark EUDRAGIT L; poly (methacrylic acid, methyl methacrylate) 1:2, 135,000, sold under the trademark EUDRAGIT S; poly (ethyl acrylate, methyl methacrylate, trimethylammonioethyl methacrylate chloride) 1:2:0.2, 150,000, sold under the trademark EUDRAGIT RL; poly(ethyl acrylate, methyl methacrylate, trimethylammonioethyl methacrylate chloride) 1:2:0.1, 150,000, sold as EUDRAGIT RS. In each case, the ratio x:y:z indicates the molar proportions of the monomer units and the last number is the number average molecular weight of the polymer. Especially preferred are cellulose acetate containing plasticizers such as acetyl tributyl citrate and ethylacrylate methylmethylacrylate copolymers such as Eudragit NE.

The foregoing materials for use as the barrier layer can be formulated with plasticizers to make the barrier layer suitably deformable such that the force exerted by the expandable layer will collapse the compartment formed by the barrier layer to dispense the liquid, active agent formulation. Examples of typical plasticizers are as follows: polyhydric alcohols, triacetin, polyethylene glycol, glycerol, propylene glycol, acetate esters, glycerol triacetate, triethyl citrate, acetyl triethyl citrate, glycerides, acetylated monoglycerides, oils, mineral oil, castor oil and the like. The plasticizers can be blended into the material in amounts of 10-50 weight percent based on the weight of the material.

The various layers forming the barrier layer, expandable layer and semipermeable layer can be applied by conventional coating methods such as described in U.S. Pat. No. 5,324,280, incorporated herein by reference in its entirety for all purposes. While the barrier layer, expandable layer and semipermeable wall have been illustrated and described for convenience as single layers, each of those layers can be composites of several layers. For example, for particular applications it may be desirable to coat the capsule with a first layer of material that facilitates coating of a second layer having the permeability characteristics of the barrier layer. In that instance, the first and second layers comprise the barrier layer. Similar considerations would apply to the semipermeable layer and the expandable layer.

The term “orifice” or “exit orifice” as used herein comprises means suitable for releasing the active agent from the dosage form. The expression includes aperture, hole, bore, pore, porous element, porous overlay, porous insert, hollow fiber, capillary tube, microporous insert, microporous overlay, and the like. The exit orifice can be formed by mechanical drilling, laser drilling, eroding an erodible element, extracting, dissolving, bursting, or leaching a passageway former from the composite wall. The exit orifice can be a pore formed by leaching sorbitol, lactose or the like from a wall or layer as disclosed in U.S. Pat. No. 4,200,098, herein incorporated by reference in its entirety for all purposes. This patent discloses pores of controlled-size porosity formed by dissolving, extracting, or leaching a material from a wall, such as sorbitol from cellulose acetate. A preferred form of laser drilling is the use of a pulsed laser that incrementally removes material from the composite wall to the desired depth to form the exit orifice.

The pharmaceutical compositions are generally formulated as sterile, substantially isotonic and in full compliance with all Good Manufacturing Practice (GMP) regulations of the U.S. Food and Drug Administration.

The osmotic devices of the present invention can optionally comprise more than one drug layer. In osmotic devices with multiple drug layers, a drug concentration gradient between the layers facilitates the achievement of an ascending drug release rate for an extended time period. For example, in one embodiment of the present invention, the osmotic dosage form comprises a first drug layer and a second drug layer, wherein the concentration of drug contained within the first layer is greater than the concentration of drug contained within the second layer, and the expandable layer is contained within a third layer. In outward order from the core of the dosage form is the first drug layer, the second drug layer and the expandable layer. In operation through the cooperation of the dosage form components, topiramate is successively released, in a sustained and controlled manner, from the second topiramate layer and then from the first topiramate layer such that an ascending release rate over an extended time period is achieved.

The release from the present invention can, for example, provide efficacious therapy over 24 hours. Dosage forms of the present invention can release topiramate from the core at a uniform zero order or uniform ascending rate, depending upon the composition of the dosage form.

Dosage forms of this invention exhibit sustained release of drug over a continuous time period that includes a prolonged time when drug is released at a uniform release rate as determined in a standard release rate assay such as that described herein. The method is practiced with dosage forms that are adapted to release the compound at various rates of release depending upon the therapeutic indications over a prolonged time period.

Although the foregoing invention has been described in detail by way of example for purposes of clarity of understanding, it will be apparent to persons skilled in the art that certain changes and modifications are comprehended by the disclosure and can be practiced without undue experimentation within the scope of the appended claims, which are presented by way of illustration not limitation.

All publications and patent documents cited above are hereby incorporated by reference in their entirety for all purposes to the same extent as if each were so individually denoted.

Each recited range includes all combinations and subcombinations of ranges, as well as specific numerals contained therein.

### EXAMPLES

### Example 1

A hard cap oral osmotic device system was manufactured for dispensing beneficial topiramate in the G.I. tract. First, an osmotic-layer formation was granulated with Glatt fluid bed granulator (FBG). One dry ingredient—NaCI was sized/screened using a Quardo mill with a 21-mesh screen at the speed set on maximum. The following dry ingredients were added into a granulator bowl: 58.75% NaCMC, 30% sized/screened NaCI, 5.0% HPMC E-5 and 1.0% red ferric oxide. The ingredients were blended in the bowl. In a separate container, the granulating solution was prepared by dissolving 5.0% HPC EF in purified water. The granulating solution was sprayed onto the fluidized powders until all of the solution is applied and the powders are granular. 0.25% Mg stearate was blended with the granules.

Second, the osmotic-layer granules and Kollidone SR were compressed into a bi-layer tablet with a tableting press or Carver press. Two hundred and seventy mg of the osmotic-layer granules were added to a 0.70 cm punch (lower punch: modified ball, upper punch: modified), tamped and then 80 mg of Kollidone SR were added onto a finally compressed under a force of about 1 metric ton into a osmotic/barrier bi-layer tablet.

Third, 40% topiramate was dissolved into 30% Cremophor EL and 30% PEG 400 using a mechanical agitator.

Next, an HMPC hard capsule (clear, size 0) was first separated into two segments (body and cap). The drug-layer composition (500 mg) was filled into the capsule body and then the osmotic/barrier tablet was placed in the filled capsule body.

Next, the membrane composition comprising 80% cellulose acetate 398-10 and 20% Pluronic F-68 was dissolved in acetone with solid content of 4% in the coating solution. The solution was sprayed onto the pre-coating assemblies in a 12″ Freud Hi-coater. The assemblies were coated with 50-100 mg of the rate-controlling membrane.

After membrane coating, the systems were dried in a Blue oven at 30° C. overnight. The 0.5 mm of orifice was drilled at the drug-layer side using a mechanical drill with drilling depth control. Each system comprises 200 mg of topiramate. By adjusting the membrane weight, the release duration of the systems can be controlled.

### Example 2

The procedure of Example 1 was repeated in this example for providing the following system. The compositions of the osmotic/barrier bilayer tablet and the rate-controlling membrane are identical to that in Example 1. But the drug-layer composition comprises, in weight percent, 60% topiramate, 20% Cremophor EL and 20% PEG 400. The dose of the system was 300 mg.

### Example 3

The following dosage ranges and drug concentrations for the oral dosage forms of the present invention are exemplary only and are proposed based on solubility study results.

Hard Cap Dosage Form:

| | | | % TPM (g/g) | | % TPM (g/g) | % TPM (g/g) |
|---|---|---|---|---|---|---|
| | | | 0.1 | % TPM (g/g) 5 | 16 | 40 |
| Capsule | Capacity | Capacity | Dosage | Dosage | Dosage | Dosage |
| Size | (mL) | (g) | (mg) | (mg) | (mg) | (mg) |
| 000 | 1.37 | 0.99 | 0.99 | 49.32 | 157.82 | 394.56 |
| 00 | 0.91 | 0.66 | 0.66 | 32.76 | 104.83 | 262.08 |
| 0el | 0.78 | 0.56 | 0.56 | 28.08 | 89.86 | 224.64 |
| 0 | 0.68 | 0.49 | 0.49 | 24.48 | 78.34 | 195.84 |
| 1 | 0.50 | 0.36 | 0.36 | 18.00 | 57.60 | 144.00 |
| 2 | 0.37 | 0.27 | 0.27 | 13.32 | 42.62 | 106.56 |
| 3 | 0.30 | 0.22 | 0.22 | 10.80 | 34.56 | 86.40 |
| 4 | 0.21 | 0.15 | 0.15 | 7.56 | 24.19 | 60.48 |
| 5 | 0.10 | 0.07 | 0.07 | 3.60 | 11.52 | 28.80 |

Soft Cap Dosage Form:

| | | | % TPM (g/g) | | % TPM (g/g) | % TPM (g/g) |
|---|---|---|---|---|---|---|
| | | | 0.1 | % TPM (g/g) 5 | 16 | 40 |
| Capsule | Capacity | Capacity | Dosage | Dosage | Dosage | Dosage |
| Size | (mL) | (g) | (mg) | (mg) | (mg) | (mg) |
| 16 | 1.22 | 0.88 | 0.88 | 43.92 | 140.54 | 351.36 |
| 14 | 1.08 | 0.78 | 0.78 | 38.88 | 124.42 | 311.04 |
| 12 | 0.89 | 0.64 | 0.64 | 32.04 | 102.53 | 256.32 |
| 10 | 0.76 | 0.55 | 0.55 | 27.36 | 87.55 | 218.88 |
| 9 | 0.66 | 0.48 | 0.48 | 23.76 | 76.03 | 190.08 |
| 8 | 0.56 | 0.40 | 0.40 | 20.16 | 64.51 | 161.28 |
| 6 | 0.42 | 0.30 | 0.30 | 15.12 | 48.38 | 120.96 |
| 5 | 0.36 | 0.26 | 0.26 | 12.96 | 41.47 | 103.68 |
| 4 | 0.30 | 0.22 | 0.22 | 10.80 | 34.56 | 86.40 |
| 3 | 0.23 | 0.17 | 0.17 | 8.28 | 26.50 | 66.24 |

Hard Cap Dosage Form:

| | | | % TPM | |
|---|---|---|---|---|
| Capsule | | Capacity | (g/g) = 45 | % TPM (g/g) = 60 |
| size | Capacity (mL) | (g) | Dosage (mg) | Dosage (mg) |
| 000 | 1.37 | 0.69 | 308.25 | 411.00 |
| 00 | 0.91 | 0.46 | 204.75 | 273.00 |
| 0el | 0.78 | 0.39 | 175.50 | 234.00 |
| 0 | 0.68 | 0.34 | 153.00 | 204.00 |
| 1 | 0.50 | 0.25 | 112.50 | 150.00 |
| 2 | 0.37 | 0.19 | 83.25 | 111.00 |
| 3 | 0.30 | 0.15 | 67.50 | 90.00 |
| 4 | 0.21 | 0.11 | 47.25 | 63.00 |
| 5 | 0.10 | 0.05 | 22.50 | 30.00 |

Soft Cap Dosage Form

| | | | % TPM | |
|---|---|---|---|---|
| Capsule | | Capacity | (g/g) = 45 | % TPM (g/g) = 60 |
| size | Capacity (mL) | (g) | Dosage (mg) | Dosage (mg) |
| 16 | 1.22 | 0.61 | 274.50 | 366.00 |
| 14 | 1.08 | 0.54 | 243.00 | 324.00 |
| 12 | 0.89 | 0.45 | 200.25 | 267.00 |
| 10 | 0.76 | 0.38 | 171.00 | 228.00 |
| 9 | 0.66 | 0.33 | 148.50 | 198.00 |
| 8 | 0.56 | 0.28 | 126.00 | 168.00 |
| 6 | 0.42 | 0.21 | 94.50 | 126.00 |
| 5 | 0.36 | 0.18 | 81.00 | 108.00 |
| 4 | 0.30 | 0.15 | 67.50 | 90.00 |
| 3 | 0.23 | 0.12 | 51.75 | 69.00 |

Hard Cap Dosage Form:

| | | | % TPM | |
|---|---|---|---|---|
| Capsule | | Capacity | (g/g) = 70 | % TPM (g/g) = 80 |
| size | Capacity (mL) | (g) | Dosage (mg) | Dosage (mg) |
| 000 | 1.37 | 0.69 | 479.50 | 548.00 |
| 00 | 0.91 | 0.46 | 318.50 | 364.00 |
| 0el | 0.78 | 0.39 | 273.00 | 312.00 |
| 0 | 0.68 | 0.34 | 238.00 | 272.00 |
| 1 | 0.50 | 0.25 | 175.00 | 200.00 |
| 2 | 0.37 | 0.19 | 129.50 | 148.00 |
| 3 | 0.30 | 0.15 | 105.00 | 120.00 |
| 4 | 0.21 | 0.11 | 73.50 | 84.00 |
| 5 | 0.10 | 0.05 | 35.00 | 40.00 |

Soft Cap Dosage Form:

| | | | % TPM | |
|---|---|---|---|---|
| Capsule | | Capacity | (g/g) = 70 | % TPM (g/g) = 80 |
| size | Capacity (mL) | (g) | Dosage (mg) | Dosage (mg) |
| 16 | 1.22 | 0.61 | 427.00 | 488.00 |
| 14 | 1.08 | 0.54 | 378.00 | 432.00 |
| 12 | 0.89 | 0.45 | 311.50 | 356.00 |
| 10 | 0.76 | 0.38 | 266.00 | 304.00 |
| 9 | 0.66 | 0.33 | 231.00 | 264.00 |
| 8 | 0.56 | 0.28 | 196.00 | 224.00 |
| 6 | 0.42 | 0.21 | 147.00 | 168.00 |
| 5 | 0.36 | 0.18 | 126.00 | 144.00 |
| 4 | 0.30 | 0.15 | 105.00 | 120.00 |
| 3 | 0.23 | 0.12 | 80.50 | 92.00 |

### Example 4

Prophetic example for preparing Topiramate Multilayer hardcap 250 mg System for ascending release rate.

A dosage form adapted, designed and shaped as an osmotic drug delivery device is manufactured as follows: drug layer 1: 4500 g of Solutol HS-15 (or for example, Gelucire 44/14) and 1500 g of Polyethylene Glycol 400 (PEG-400) are added to a jacketed mixing tank with the tank being pre-heated to 40° C. (50° C. for Gelucire 44/14), 4000 g of topiramate is then added into the mixing tank after the excipients in the tank liquidated and mixed well. Mixing continues till all components in the tank become a homogenous mixture. Upon cooling, the formulation will be solidified and become semi-solid in nature.

Next, the drug layer 2 is prepared as follows: 3000 g of Solutol HS-15 (or Gelucire 44/14) and 1000 g of Polyethylene Glycol 400 (PEG-400) are added to a mixer with the Solutol HS-15 (or Gelucire 44/14) being pre-melted. 6000 g of topiramate is then added into the mixing tank after the excipients in the tank liquidated and mixed well. Mixing continues till all components in the tank become a homogenous mixture. Upon cooling, the formulation will be solidified and become semi-solid in nature.

Next, a bi-layer osmotic engine is prepared as follows: preparation of a push granulation followed by compression of push granulation and barrier materials (50% kollidone and 50% of fine wax) into a bi-layer osmotic engine (300 mg of push granulation and 150 barrier materials) on multilayer Korsch press. The diameter of the bi-layer engine should be well defined so that the bilayer engine will tightly fit into a zero-size HPMC or gelatin hard capsule. A push granulation is prepared as following: first, a binder solution is prepared. 15.6 kg of polyvinylpyrrolidone identified as K29-32 having an average molecular weight of 40,000 is dissolved in 104.4 kg of water. Then, 24 kg of sodium chloride and 1.2 kg of ferric oxide are sized using a Quadro Comil with a 21-mesh screen. Then, the screened materials and 88.44 kg of Polyethylene oxide (approximately 2,000,000 molecular weight) are added to a fluid bed granulator bowl. The dry materials are fluidized and mixed while 46.2 kg of binder solution is sprayed from 3 nozzles onto the powder. The granulation is dried in the fluid-bed chamber to an acceptable moisture level. The coated granules are sized using a Fluid Air mill with a 7-mesh screen. The granulation is transferred to a tote tumbler, mixed with 15 g of butylated hydroxytoluene and lubricated with 294 g magnesium stearate.

Next, two liquid formulation layers and bi-layer osmotic engine are assembled into a hardcap delivery system on hardcap assembly machine. The manufacturing process is described as follows: 250 mg of pre-liquidated formulation layer 1 is filled into a half-body HPMC capsule (zero-size HPMC capsules are de-capped before filling process begins). Allow the formulation layer 1 to be solidified upon a quick cooling process. Next, 250 mg of pre-liquidated formulation layer 2 is filled into the capsule on the top of the layer 1. Allow the formulation layer 2 to be solidified upon a quick cooling process. Next, the bilayer osmotic engine is inserted into the capsule on the top of the formulation layer 2.

The multilayer arrangements are coated with a semi-permeable wall. The wall forming composition comprises 99% cellulose acetate having a 39.8% acetyl content and 1% polyethylene glycol comprising a 3.350 viscosity-average molecular weight. The wall-forming composition is dissolved in an acetone:water (95:5 wt:wt) co solvent to make a 5% solids solution. The wall-forming composition is sprayed onto and around the multilayer arrangements in a pan coater until approximately 39 mg of membrane is applied to each tablet.

Next, one 30 mil (0.76 mm) exit passageway is laser drilled through the semi-permeable wall to connect the drug layer with the exterior of the dosage system. The residual solvent is removed by drying for approximately 48-72 hours as 40 C.° and ambient humidity.

Next, the drilled and dried systems are color overcoated. The color overcoat is a 12% solids suspension of Opadry in water. The color overcoat suspension is sprayed onto the drug-overcoated systems until an average wet coated weight of approximately 25 mg per system is achieved.

The dosage form produced by this manufacture is designed to deliver 250 mg of topiramate in an ascending delivery pattern.

### Example 5

### Drug Solubility in Various Carriers

45% topiramate in 100% of PEG400 was found to be completely soluble which allows for the preparation of a hard cap dosage form with 200-250 mg dosage using a 0 size capsule.—20-30% topiramate in 90/10, 80/20 or 70/30 PEG 400/Cremophor EL was completely soluble which allows for the preparation of a hard cap dosage form with 100-150 mg topiramate using a 0 size capsule.

1. A dosage form for controlled release delivery of topiramate in a liquid formulation, the dosage form comprising:
a. a semipermeable wall permeable to the passage of an exterior biological fluid and substantially impermeable to the passage of drug formulation, the wall surrounding and forming a compartment containing a plurality of layers wherein at least one layer is a drug layer comprising topiramate solubilized in a nonaqueous liquid carrier and at least one other layer is expandable; and
b. an orifice in the semipermeable wall connecting the exterior of the dosage form and the topiramate formulation for delivering topiramate from the dosage form to the environment.

2. The dosage form of claim 1, wherein the liquid formulation comprises a lipophilic solvent, a surfactant, a hydrophilic solvent or a combination thereof.

3. The dosage form of claim 2, wherein the hydrophilic solvent is a liquid polymer.

4. The dosage form of claim 1, wherein the expandable layer is an osmotic layer.

5. The dosage form of claim 1 wherein the expandable layer comprises a fluid-expandable polymer.

6. The dosage form of claim 1 wherein the compartment further comprises a barrier layer.

7. The dosage form of claim 6, wherein the barrier layer is impermeable to water.

8. The dosage form of claim 6 wherein the expandable layer is longitudinally compressed.

9. The dosage form of claim 7, wherein the topiramate layer is encased in a soft capsule and in outward order from the capsule is a barrier layer, an expandable layer, and the semipermeable wall.

10. The dosage form of claim 9, wherein the barrier layer is formed as a coating on the capsule.

11. The dosage form of claim 10, wherein the expandable layer is formed as an osmotic layer coated on the barrier layer.

12. The dosage form of claim 11, wherein the semipermeable wall is formed as a coating on the osmotic layer.

13. The dosage form of claim 1, wherein the topiramate layer, the barrier layer, and the expandable layer are encased in a hard capsule, the barrier layer separating the topiramate layer from the expandable layer; and surrounding the hard capsule is the semipermeable wall.

14. The dosage form of claim 13 wherein the expandable layer is formed as an osmotic layer compressed on the barrier layer.

15. The dosage form of claim 1, wherein the liquid formulation comprises a hydrophilic solvent.

16. The dosage form of claim 15, wherein the hydrophilic solvent is a liquid polymer.

17. The dosage form of claim 16, wherein the liquid polymer is polyethylene glycol.

18. The dosage form of claim 13 further comprising a second layer comprising topiramate solubilized in a liquid carrier.

19. The dosage form of clam 18, wherein the second layer comprises a greater concentration of topiramate than the first layer.

20. The dosage form of claim 1 wherein the liquid formulation is a solution or suspension.

21. The dosage form of claim 1 wherein the liquid formulation is a self emulsifying formulation.

22. The dosage form of claim 21 wherein the self emulsifying formulation is lipid based.

23. The dosage form of claim 1 wherein the dosage form comprises less than about 100 mg of topiramate.

24. The dosage form of claim 1 wherein the dosage form comprise from about 100 mg to about 800 mg of topiramate.

25. The dosage form of claim 1 wherein the dosage form comprises from about 1 mg to about 600 mg of topiramate.

26. The dosage form of claim 1 wherein the dosage form comprises from about 1 mg to about 400 mg of topiramate.

27. The dosage form of claim 1 wherein the dosage form comprises from about 1 mg to about 300 mg of topiramate.

28. The dosage form of claim 1 wherein the dosage form comprise from about 10 mg to about 600 mg of topiramate.

29. The dosage form of claim 1 wherein the dosage form comprise from about 25 mg to about 400 mg of topiramate.

30. The dosage form of claim 1 wherein the dose of topiramate is between about 0.1% to about 60% by weight of the dosage form.

31. The dosage form of claim 1 wherein the liquid carrier is between about 30% to about 50% by weight of the dosage form.

32. The dosage form of claim 1 wherein the drug layer comprises from about 10% to about 60% of topiramate and about 40% to about 90% of a liquid carrier.

33. The dosage form of claim 1 wherein the drug layer comprises 40% to about 60% of topiramate and about 60% to about 40% of a hydrophilic liquid solvent.

34. The dosage form of claim 33 wherein the hydrophilic liquid solvent is PEG400.

35. The dosage form of claim 1 wherein the drug layer comprises about 40% topiramate, about 30% of a surfactant, and about 30% of a hydrophilic liquid solvent.

36. The dosage form of claim 35 wherein the surfactant is cremophor EL or solutol and the hydrophilic liquid solvent is PEG400.

37. The dosage form of claim 1 wherein the drug layer comprises about 60% topiramate, about 20% of a surfactant, and about 20% of a hydrophilic liquid solvent.

38. The dosage form of claim 37 wherein the surfactant is cremophor EL or solutol and the hydrophilic liquid solvent is PEG400.

39. The dosage form of claim 1 wherein the liquid carrier comprises a surfactant and hydrophilic solvent in a ratio of 60% surfactant to 40% hydrophilic liquid solvent.

40. The dosage form of claim 39 wherein the surfactant is cremophor EL or solutol and the hydrophilic solvent is PEG400.

41. A method for controlled release delivery of a topiramate in a liquid formulation, the method comprising administering a sustained release, liquid formulation dosage form of topiramate to a subject, the dosage form comprising:
a. a semipermeable wall permeable to the passage of an exterior biological fluid and substantially impermeable to the passage of drug formulation, the wall surrounding and forming a compartment containing a plurality of layers wherein at least one layer is a drug layer comprising topiramate solubilized in a liquid carrier and at least one other layer is expandable; and
b. an orifice in the semipermeable wall connecting the exterior of the device and the topiramate formulation for delivering topiramate from the device to the environment.

42. The dosage form of claim 41, wherein the liquid formulation comprises a lipophilic solvent, a surfactant, a hydrophilic solvent or a combination thereof.

43. The dosage form of claim 41, wherein the liquid carrier comprises a hydrophilic solvent. liquid polymer.

44. The dosage form of claim 43, wherein the hydrophilic solvent is a liquid polymer.

45. The method of claim 41 wherein the expandable layer is an osmotic layer.

46. The method of claim 41 wherein the compartment further contains a barrier layer.

47. The dosage form of claim 46, wherein the topiramate layer is encased in a soft capsule and in outward order from the capsule is a barrier layer, an expandable layer, and the semipermeable wall.

48. The dosage form of claim 46, wherein the topiramate layer, the barrier layer, and the expandable layer are encased in a hard capsule, the barrier layer separating the topiramate layer from the expandable layer; and surrounding the hard capsule is the semipermeable wall.

49. The method of claim 41 wherein the release rate of topiramate is zero order.

50. The method of claim 48 wherein the release rate of topiramate is ascending.

51. The method of claim 48 further comprising a second layer comprising topiramate solubilized in a nonaqueous liquid carrier.

52. The method of clam 51, wherein the second layer comprises a greater concentration of topiramate than the first layer.

53. The method of claim 52, wherein topiramate is released successively from the second layer and then from the first layer.

54. The method of claim 41 wherein the dosage form comprises less than about 100 mg of topiramate.

55. The method of claim 41 wherein the dosage form comprises from about 100 mg to about 600 mg of topiramate.

56. The method of claim 41 wherein the dosage form comprise from about 1 mg to about 400 mg of topiramate.

57. The method of claim 41 wherein the dosage form comprise from about 1 mg to about 300 mg of topiramate.

58. The method of claim 41 wherein the dosage form comprise from about 15 mg to about 600 mg of topiramate.

59. The method of claim 41 wherein the dosage form comprise from about 25 mg to about 400 mg of topiramate.

60. The method of claim 41 wherein the dose of topiramate is between about 0.1% to about 60% by weight of the dosage form.

61. The method of claim 41 wherein the liquid carrier is between about 30% to about 50% by weight of the dosage form.

62. The method of claim 41 wherein the drug layer comprises about 10% to about 60% of topiramate and about 40% to about 60% of a liquid carrier.

63. The method of claim 41 wherein the drug layer comprises from about 40% to about 60% of topiramate and from about 60% to about 40% of a hydrophilic liquid solvent.

64. The method of claim 63 wherein the hydrophilic liquid solvent is PEG400.

65. The method of claim 41 wherein the drug layer comprises about 40% topiramate, about 30% of a surfactant, and about 30% of a hydrophilic liquid solvent.

66. The method of claim 65 wherein the surfactant is cremophor EL or solutol and the hydrophilic solvent is PEG400.

67. The method of claim 41 wherein the drug layer comprises about 60% topiramate, about 20% of a surfactant, and about 20% of a hydrophilic liquid solvent.

68. The method of claim 67 wherein the surfactant is cremophor EL or solutol and the hydrophilic liquid solvent is PEG400.

69. The method of claim 41 wherein the liquid carrier comprises a surfactant and hydrophilic liquid solvent in a ratio of 60% surfactant to 40% hydrophilic liquid solvent.

70. The method of claim 69 wherein the surfactant is cremophor EL or solutol and the hydrophilic lqiud solvent is PEG400.

71. A method for administering topiramate in liquid form to a subject, the method comprising orally administering a dosage form comprising:
a. a semipermeable wall permeable to the passage of an exterior biological fluid and substantially impermeable to the passage of drug formulation, the wall surrounding and forming a compartment containing a plurality of layers wherein at least one layer is a drug layer comprising topiramate solubilized in a liquid carrier and at least one other layer is expandable; and
b. an orifice in the semipermeable wall connecting the exterior of the device and the topiramate formulation for delivering topiramate from the device to the environment to a subject.

72. The method of claim 71 wherein the subject suffers from seizures.

73. The method of claim 71 wherein the subject suffers from mood disorders.

74. The method of claim 71 wherein the subject is obese.

75. The method of claim 71 wherein the subject suffers from diabetes.

76. The method of claim 71 wherein the subject has an eating disorder.

77. The method of claim 71 wherein the subject suffers from migraines.
